(19) United States
(12) Patent Application Publication
(10) Pub. No.: US 2001/0032011 A1
(43) Pub. Date: Oct. 18, 2001

(54) Expandable stent with array of relief cuts

(76) Inventor: Ulf Harry Stanford, Incline Village, NV (US)

Correspondence Address: Bruce H. Johnsonbaugh, Eckhoff, Hoppe, Slick, Mitchell & Anderson, Suite 3125, 333 Market Street, San Francisco, CA, 94111, US

(21) Appl. No.: 09/774,760

(22) Filed: Jan. 30, 2001

Related U.S. Application Data
(63) Continuation-in-part of application No. 09/357,699, filed on Jul. 20, 1999.

(51) Int. Cl. A61F 2/06

(52) U.S. Cl. 623/1.15

(57) ABSTRACT

An array of relief cuts is formed at the flexion points of an expandable stent. The relief cuts are small enough to preserve the columnar compressive strength of the stent. The relief cuts allow a balloon expandable stent to be expanded with less balloon pressure. The relief cuts allow the use of wider and thinner material in the stent, giving greater radio-opacity and vessel coverage while simultaneously reducing the stent profile and increasing laminar blood flow through the stented vessel. Patterns of relief cuts may be placed in stents to achieve controlled, non-uniform expansion and which, in some instances, allow the stent to bend easily in a given direction to facilitate deployment in curved arteries or other body lumens.

### CROSS REFERENCE TO RELATED APPLICATION

This is a continuation-in-part of U.S. patent application Ser. No. 09/357,699 filed Jul. 20, 1999 and entitled EXPANDABLE STENT.

### BACKGROUND AND BRIEF SUMMARY OF THE INVENTION

The present invention relates generally to balloon expandable and self-expanding stents. More particularly, the present invention provides an array of relief cuts for use in a variety of stent designs. According to the present invention, the relief cuts are strategically placed to allow the stent to expand more easily, requiring less pressure for the expansion, without any significant loss of columnar compressive strength of the members making up the stent. The present invention allows the use of wider and thinner material, resulting in a stent with greater radio-opacity, and resulting in a thinner walled stent allowing maximum blood flow through the artery or other lumen.

The prior art stent designs typically use stent members or struts having circular cross sections as illustrated in FIGS. 4B, 4C and 4F or generally square cross sections as illustrated in FIG. 4H. FIG. 4A illustrates a typical prior art diamond-shaped cell configuration utilizing the cross sections illustrated in FIGS. 4B, 4C, 4F and 4H. The prior art configurations illustrated in FIGS. 4A-4C and 4H have several disadvantages. First, as the stents are downsized for use in smaller vessels, the circular or cross sections of the stent material tends to effectively reduce the cross-sectional area of the artery or other body lumen which is capable of achieving low turbulence or laminar blood flow. A second disadvantage of this typical prior art design is that, in many instances, particularly as the stent size is reduced, the radio-opacity of the stent, i.e., the capability of the stent to be viewed by the surgeon, is reduced. A third disadvantage is limited coverage of the vessel wall by the stent. The present invention is designed to overcome these problems by providing a wider and thinner material, as illustrated generally in FIGS. 4D, 4E and 4G, which provides enhanced radio-opacity, enhanced vessel wall coverage but at the same time providing a stent that will expand as easily as the prior art stent. For example, the stent, according to the present invention, as illustrated in FIGS. 4D, 4E and 4G, is designed to expand under the same balloon pressure as the stent designs illustrated in FIGS. 4A-4C and 4H.

The prior art also includes International Application No. PCT/US99106136 published Oct. 7, 1999 as publication No. WO 99/49810. That published application discloses a “double-strut” stent configuration with a plurality of closed cells wherein each member of the cell configuration is slotted throughout its entire length. The inherent weakness of this design is that each fully slotted cell member becomes significantly weakened by the use of slots extending throughout its entire length. The compressive strength of each member is substantially weakened as shown by the “critical load” analysis as an “Euler column” as established mathematically by Leonard Euler. The removal of approximately one-third of the material along the center of a column and throughout the entire length of the column greatly reduces its ability to support a compressive load, i.e., its hoop strength in the case of a stent.

In contrast to the “double-strut” design described in the above-identified PCT International application, the present invention utilizes an array of strategically placed relief cuts. Each individual relief cut has a relatively short length in order to preserve the ability of the member to retain its columnar compressive strength.

It is also known in the prior art to electro-polish portions of a stent to reduce the cross-sectional area of a stent member so that less balloon pressure is required to expand the stent. The electro-polishing technique is expensive and has the inherent weakness of reducing the radio-opacity of the polished portion of the stent. It is important to maximize the radio-opacity of the stent, especially as smaller stents are used in smaller arteries and other body lumens. The present invention avoids the cost and disadvantages of electro-polishing; furthermore, the present invention inherently allows the use of wider and thinner, more radio-opaque stent members.

Another significant problem with most prior art stent designs arises when a stent is placed in a curved section of an artery (or other lumen). As the balloon is expanded, the stent tends to straighten, causing the curved portion of the artery (or other lumen) to straighten and sometimes rupturing the vessel wall. The present invention facilitates expansion of a stent in a curved artery by using curved balloons and applying relief cuts in selected, predetermined patterns to the stent. The relief cuts reduce the tendency of the stent to straighten as the stent and balloon are expanded.

A further limitation of prior art stents is that the typical stent expands at a uniform rate as balloon pressure is applied. There are many practical instances where a controlled non-uniform expansion of a stent would be a significant advantage.

Another significant aspect of the present invention is that selective placement of an array of relief cuts on a stent allows the stent to expand in a predetermined and controlled non-uniform fashion. For example, placing an array of relief cuts only in the longitudinal center region of the stent causes the center region of the stent to expand first before the distal and proximal regions of the stent expand. As another example, relief cuts can be utilized only at the distal and proximal end regions of the stent, which causes the end regions to expand first, with the central region of the stent expanding last. As a further example, relief cuts may be applied in various patterns to cause stents to act differently; some patterns allow stents to be used better in curved and tapered vessels, some patterns allow one or both ends of the stent to be “flared,” and some patterns allow the stent to bend more easily in a given direction.

Another advantage of the present invention is that the relief cuts may be applied to a variety of existing and commercially successful balloon expandable and self-expanding stent designs. The use of the relief cuts as described and claimed herein can quickly provide existing commercial stents with most, if not all, of the advantages of the present invention.

It is therefore a primary object of the present invention to provide an array of relief cuts in a balloon expandable stent to allow the stent to expand more easily and with less pressure, without any significant loss of the columnar compressive strength of the stent members in which the cuts are formed.

Another object of the invention is to provide an array of relief cuts in prior art stent designs to allow those stents to expand more easily and with less pressure than is the case in the absence of relief cuts.

Still another object of the invention is to provide a balloon expandable and self-expandable stent design having an array of relief cuts which in turn allows the use of wider and thinner members in the stent to increase the radio-opacity and vessel wall coverage of the stent, while using thinner wall members.

Still a further object of the invention is to provide an array of relief cuts which not only allows the use of wider members, but also allows the use of thinner wall stents, thereby increasing the effective inner diameter of arteries and other lumens carrying those stents. The use of thinner walled stents minimizes the profile or cross section of the stent and provides more clearance in inserting and deploying the stent.

A still further object of the invention is to provide selective placement of one or more arrays of relief cuts to a stent, which allows the stent to expand in a predetermined and controlled non-uniform fashion. This feature allows a stent to be custom designed to an artery to better support the arterial wall and to seal end leaks.

Other objects and advantages of the present invention will become apparent from the following description and the drawings wherein:

### BRIEF DESCRIPTION OF THE DRAWINGS

FIGS. 1A-E are schematic representations showing relief cuts according to the present invention as applied to a generally diamond-shaped prior art stent cell configuration;

FIG. 2 is a schematic representation illustrating how the relief cuts of the present invention may be applied to a prior art “butterfly” stent cell configuration;

FIG. 3 is a schematic representation showing how the relief cuts of the present invention may be applied to a prior art stent configuration having an undulating or sinusoidal-type cell configuration;

FIGS. 4A-4C and 4F illustrate a prior art, diamond-shaped stent cell, with FIGS. 4B and 4C showing the cross section of the stent of FIG. 4A;

FIGS. 4D, 4E and 4G illustrate how the present invention as applied to the diamond-shaped cell of FIGS. 4A-4C and 4E achieves a wider, thinner stent, with a larger interior diameter allowing less turbulent blood flow;

FIG. 4H illustrates the cross section of a typical prior art stent having a generally square shape with rounded corners formed by electro-polishing;

FIGS. 5A-5D illustrate how the relief cuts of the present invention may be applied strategically to the distal and proximal ends of a stent, thereby causing those ends of the stent to expand before the central portion of the stent expands;

FIGS. 6A-6C are schematic representations illustrating how a pattern of relief cuts placed only in the central portion of a stent will cause that center portion of the stent to expand before the end portions;

FIG. 7 is a schematic representation showing how the relief cuts of the present invention as applied to only the central portion of a prior art stent configuration will cause a predetermined, controlled, uneven expansion of the stent;

FIGS. 8A and 8B are schematic illustrations representing how the present invention could be utilized to seal off the blood flow to and kill a cancerous tumor;

FIG. 9 is a schematic representation showing how relief cuts of the present invention may be used in a prior art stent having a single strand serpentine-shaped stent, illustrating how the present invention is usable in prior art stents which do not have closed cell configurations;

FIG. 10 is a schematic representation of an alternate embodiment of the invention, showing transversely spaced relief cuts for use primarily on stents having relatively wide members;

FIG. 11A illustrates schematically how relief cuts may be patterned for use on a stent to be placed in a curved artery or other body lumen wherein no relief cuts are placed in the central region of the stent;

FIG. 11B is a section on the line 11B-11B of FIG. 11A;

FIG. 12A is a schematic representation showing still another way in which relief cuts may be patterned on a stent for use in a curved artery or other body lumen;

FIG. 12B is a sectional view on the line 12B-12B of FIG. 12A;

FIG. 13A shows another embodiment wherein relief cuts are placed only in the central region of a stent and around the entire periphery of the stent for use in a curved artery and FIG. 13B is a sectional view on the line 13B-13B of FIG. 13A;

FIG. 14A is yet another embodiment showing relief cuts applied only to the central region of the stent for use in a curved artery wherein the relief cuts are placed away from the inside radius of curvature of the artery;

FIG. 14B is a section on the line 14B-14B of FIG. 14A;

FIGS. 15A and 15B are schematic illustrations showing how the present invention may be used with bifurcated stents;

FIGS. 16A and 16B are schematic illustrations showing how the present invention may be used in an irregularly shaped artery (or other lumen);

FIGS. 17A and 17B are schematic illustrations showing one way in which the present invention may be utilized in an artery (or other lumen) having a branch artery (or lumen) at or near the deployment site of the stent;

FIGS. 18A and 18B are schematic illustrations showing a second way in which the present invention may be utilized in an artery (or other lumen) having a branch artery (or lumen) at or near the deployment site of the stent; and

FIGS. 19A and 19B illustrate how the present invention may be utilized in a stent having cells which expand in different directions as the stent expands.

### DETAILED DESCRIPTION OF THE DRAWINGS

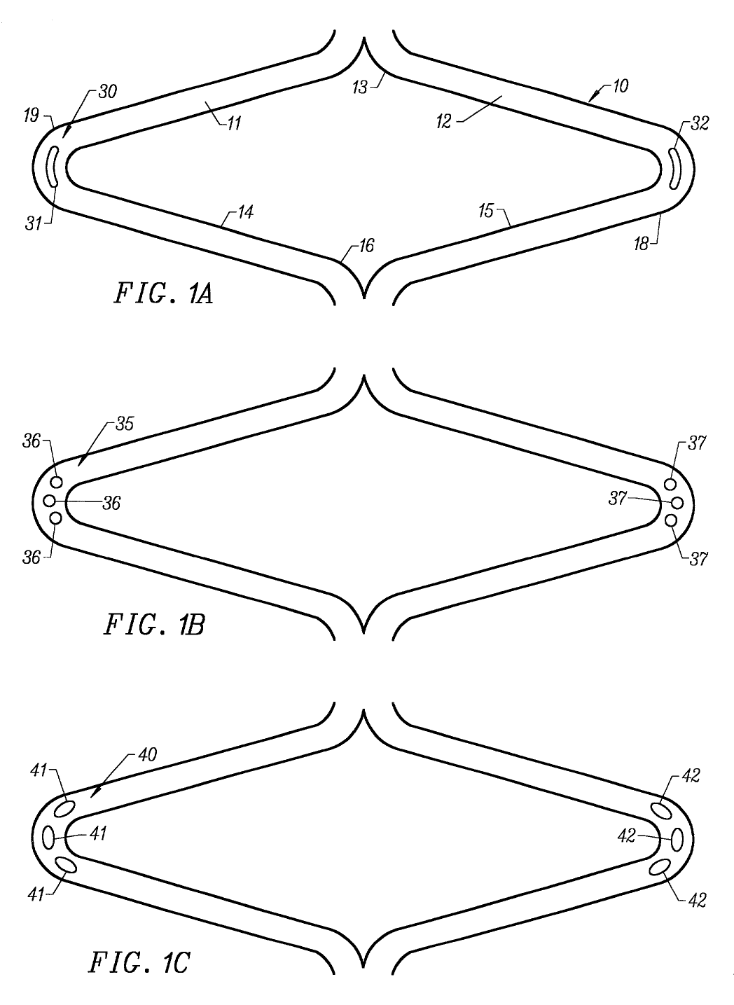

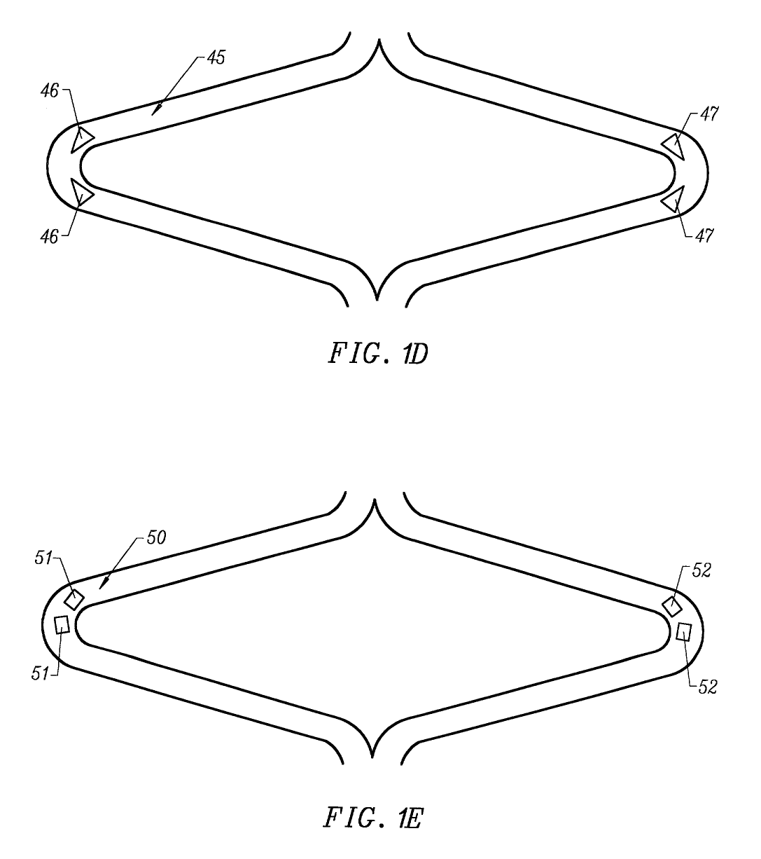

FIGS. 1A-1E illustrate how a conventional, diamond-shaped stent cell 10 may be provided with relief cuts of various shapes according to the present invention. FIG. 1A illustrates a diamond-shaped cell 10 having upper members 11 and 12 connected at their intersection 13 and lower members 14 and 15 connected at their intersection 16. As the stent cell 10 expands, members 12 and 15 flex or bend relative to each other at their connection point 18. Similarly, members 11 and 14 bend or flex relative to each other about their connection point or apex 19. Connection points 18 and 19 extend along the longitudinal axis of the stent.

According to the present invention, a plurality of relief cuts shown generally as 30 includes first and second cuts 31 and 32, each formed either in the apexes or connections between members or at the point in which the primary amount of flexing or bending occurs to allow the stent to expand. The relief cuts of the present invention may also be utilized in stents which do not use a closed cell configuration, as described below. The relief cuts 31 and 32 illustrated in FIG. 1A are arcuate cuts that extend completely through the particular members of stent 10. Placement of one or more of the relief cuts 31 and 32 at or near at least one designed flexion or bending point of the particular stent configuration allows the stent to expand in response to considerably less balloon pressure.

FIG. 1B illustrates the same stent cell configuration as FIG. 1A but illustrates a second form of relief cuts 35 which include a plurality of circular cuts 36 and 37, preferably positioned at those points of the stent design which are intended to flex or bend to allow the stent to expand. FIG. 1C illustrates a third embodiment wherein the relief cuts 40 are elliptical relief cuts 41 and 42.

FIG. 1D illustrates a fourth type of relief cut 45 which includes a plurality of triangular shaped cuts 46 and a second plurality of triangular cuts 47. Applicant believes that, although the triangular relief cuts would perform the intended function of allowing the stent to expand with less balloon pressure, those are not the preferred designs. The applicant believes the designs having sharp corners in the relief cuts may eventually cause cracking of the stent material as the stent expands.

FIG. 1E illustrates a fifth form of relief cut 50 including a first plurality 51 and a second plurality 52 of generally rectangular or square shaped cuts. Again, these are less preferable than the rounded cuts illustrated in FIGS. 1A-C but, nevertheless, are within the scope of the invention.

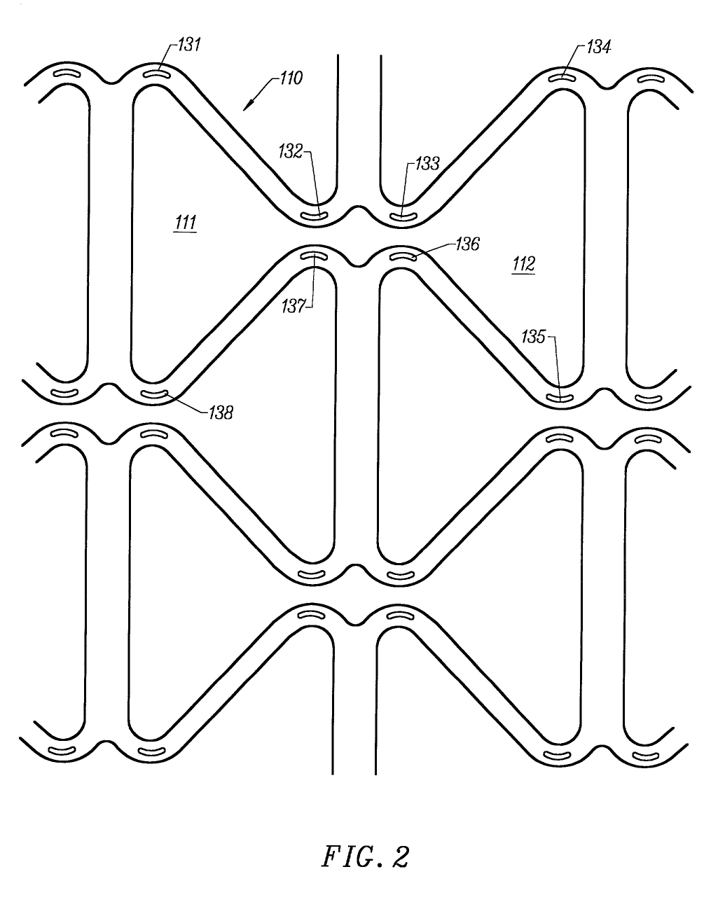

FIG. 2 illustrates a second stent cell configuration 110 having a “butterfly” shape including a first butterfly wing 111 and a second butterfly wing 112. A plurality of arcuate relief cuts 131-138 are formed strategically at those places of the cell configuration wherein maximum bending and flexing is intended to occur between the individual members of the cell configuration 110 as the stent expands. FIG. 2 illustrates the arcuate slotted relief cut. Alternately, the circular or elliptical relief cuts illustrated in FIGS. 1B and 1C could be utilized in the cell configuration shown in FIG. 2.

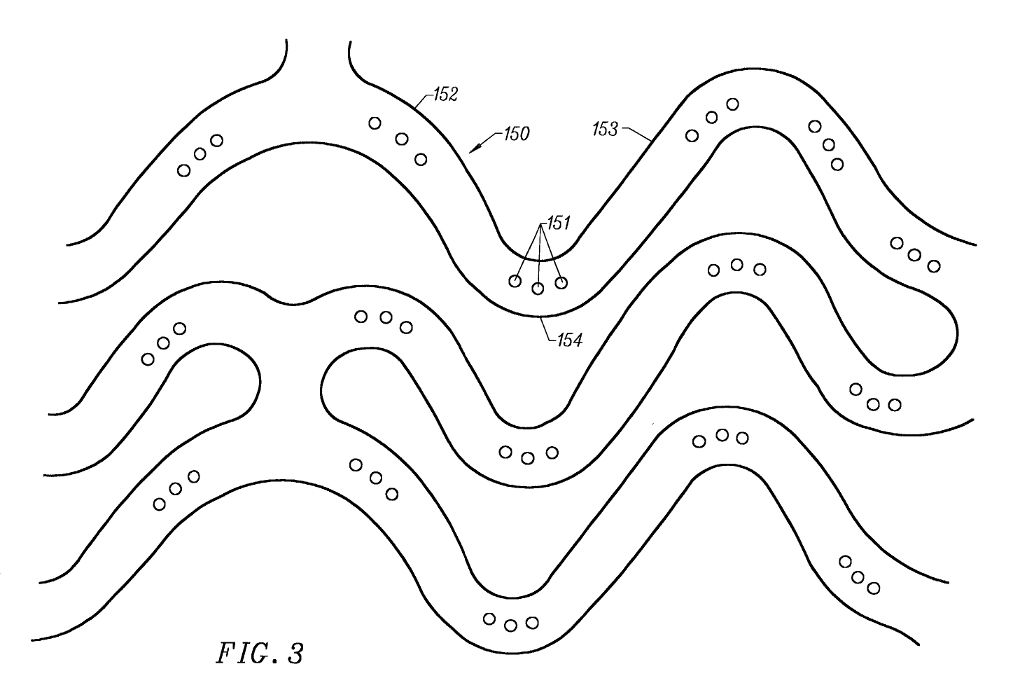

FIG. 3 illustrates the application of the present invention to a third type of prior art cell configuration 150. Cell 150 is generally a sinusoidal wall cell configuration. Relief cuts 151 are formed between adjacent members 152 and 153. Members 152 and 153 are intended to flex or bend at their connection point 154 as the stent cell 150 expands. Relief cuts 151, according to the present invention, are a series of round holes placed at this region of maximum flexion or bending to allow the stent to expand in response to reduced balloon pressure.

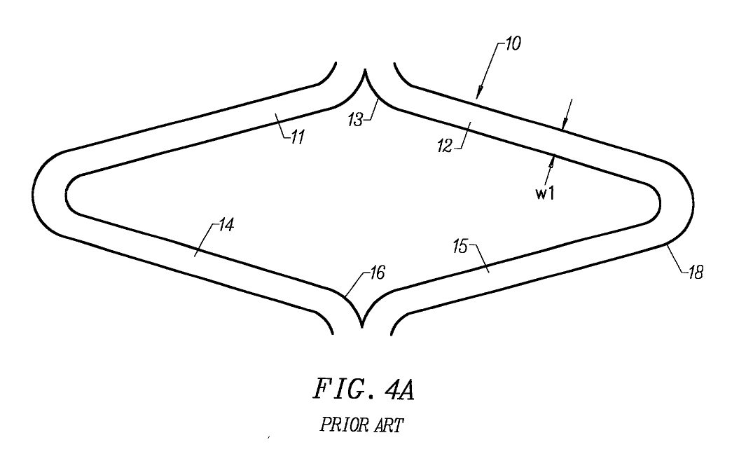

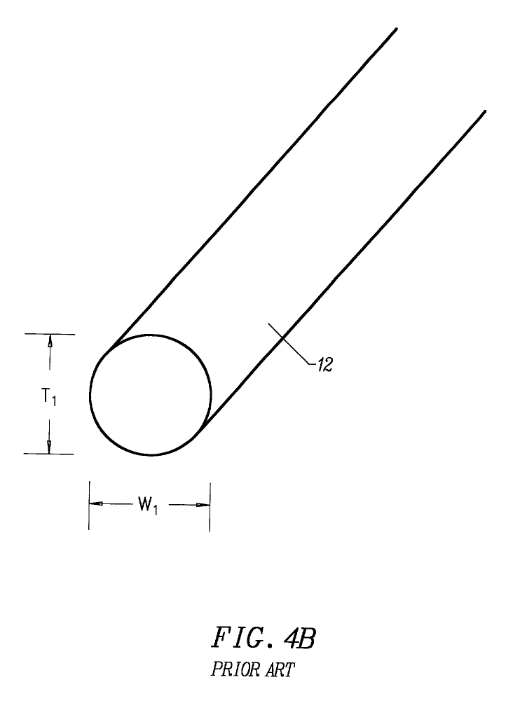

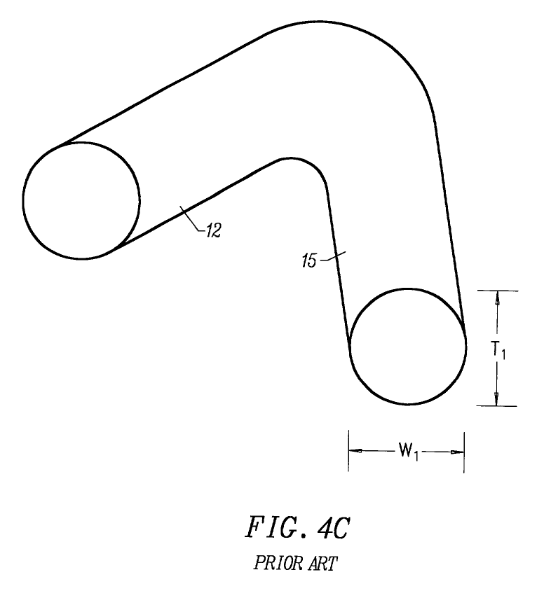

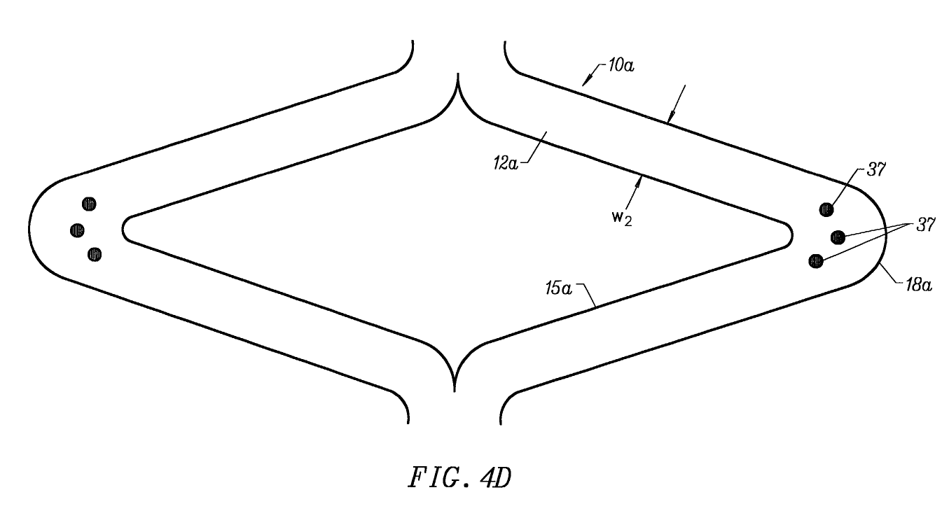

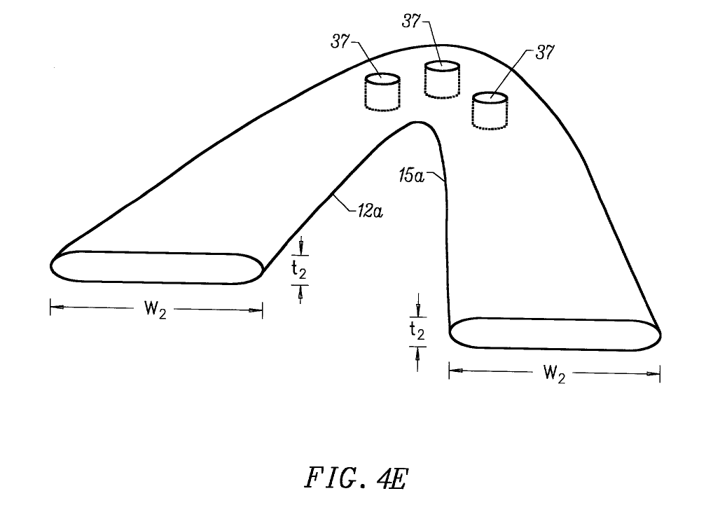

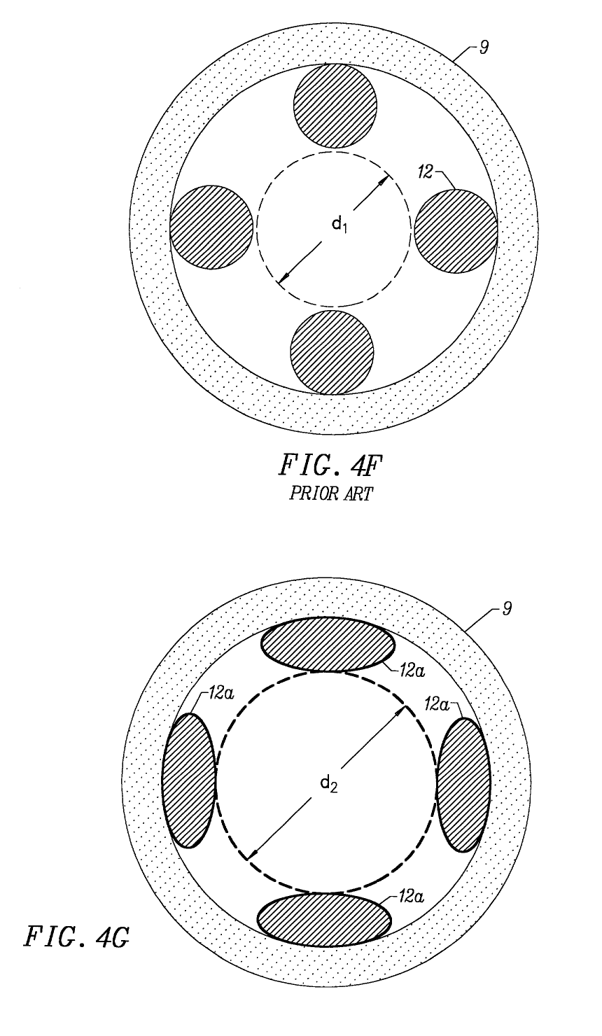

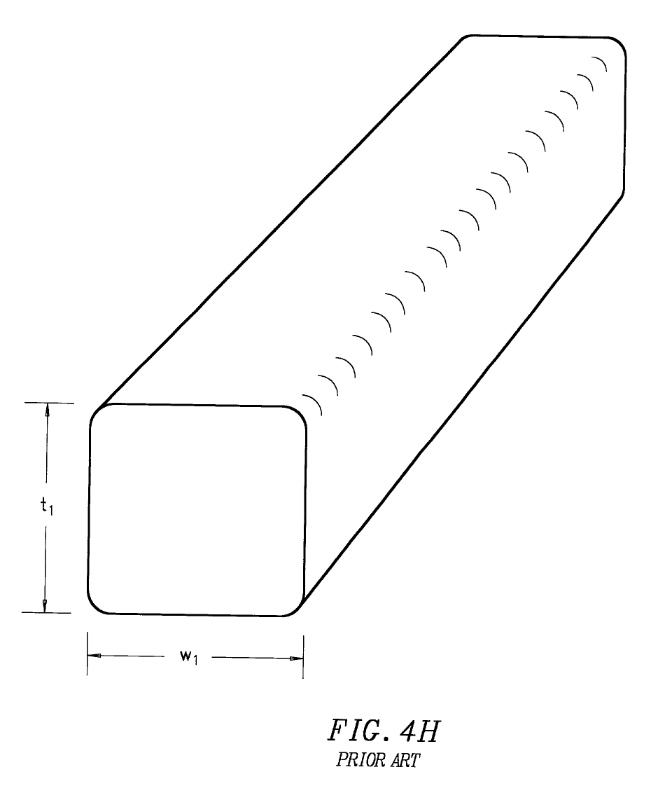

FIGS. 4A-4H are intended to illustrate how the present invention can be applied to a prior art cell configuration, and these figures illustrate some of the significant advantages that the present invention provides. FIGS. 4A-4C and 4H illustrate the prior art diamond-shaped cell configuration 10 illustrated in FIG. 1. As shown in FIG. 4A, the cell configuration is shown as it is used in the prior art, i.e., without any of the relief cuts of the present invention. FIGS. 4B and 4C show one typical cross section of members 12 and 15 as being of circular cross section and having a thickness t₁ and width w₁ that are equal to each other. FIG. 4H illustrates a second typical prior art cross section as generally square with rounded corners formed by electro-polishing, having a thickness t₁ and width w₁. FIG. 4F shows in exaggerated fashion a cross section of an artery 9 in which a stent having the cell configuration shown in FIG. 4A has been placed. Member 12 is illustrated having a circular cross section and supporting vessel wall 9. As shown in FIG. 4F, the central unobstructed portion of artery 9 has a diameter d, which is the diameter of the stented artery capable of supporting low turbulence or laminar blood flow, for a given degree of expansion of artery 9.

FIGS. 4D, 4E and 4G illustrate a modified diamond-shaped cell configuration 10a incorporating the present invention. A series of circular relief cuts 37 are shown at the intersection 18a of cell members 12a and 15a and illustrated best in FIG. 4E and are much wider and much thinner than the prior art cross sections shown in FIGS. 4B, 4C and 4H. The width w₂ is considerably greater than the width w₁ of members 12 and 15; width W₂ is also greater than the thickness t₂ of the modified cell configuration 10a. Width w₂ is preferably between 1.5 and 5 times as great as thickness t₂. However, it is significant to note that, because of the presence of relief cuts 37, the modified cell configuration 10a will expand in response to the same balloon pressure or less balloon pressure required to expand the prior art cell configuration 10. The presence of relief cuts 37 in the cell configuration allows the use of much wider and thinner members 12a and 15a of the stent. A further significant advantage is illustrated in FIG. 4G. Each stent member 12a has the general configuration illustrated in FIG. 4E and is shown in place within the same arterial wall 9 expanded to the same extent as illustrated in FIG. 4F. The reduced thickness of each stent member 12a allows a larger diameter d₂ which represents the unobstructed inner diameter of the stented artery capable of supporting less turbulent blood flow. Any increase in this diameter is very significant, particularly as smaller diameter or diseased vessels are considered. The effective volume of less turbulent laminar blood flow varies with the square of the diameter d₂ so that the difference illustrated between FIGS. 4F and 4G represents an approximately 50% increase in theoretical volumetric low turbulence, laminar blood flow in the stented artery for the same degree of expansion. Reduction of turbulence provides the added benefit of reduced blood clotting, since turbulent blood flow tends to create blood clots. As noted above, the increased width of the stent members increases radio-opacity as well as increases contact with the vessel wall as illustrated in FIG. 4G.

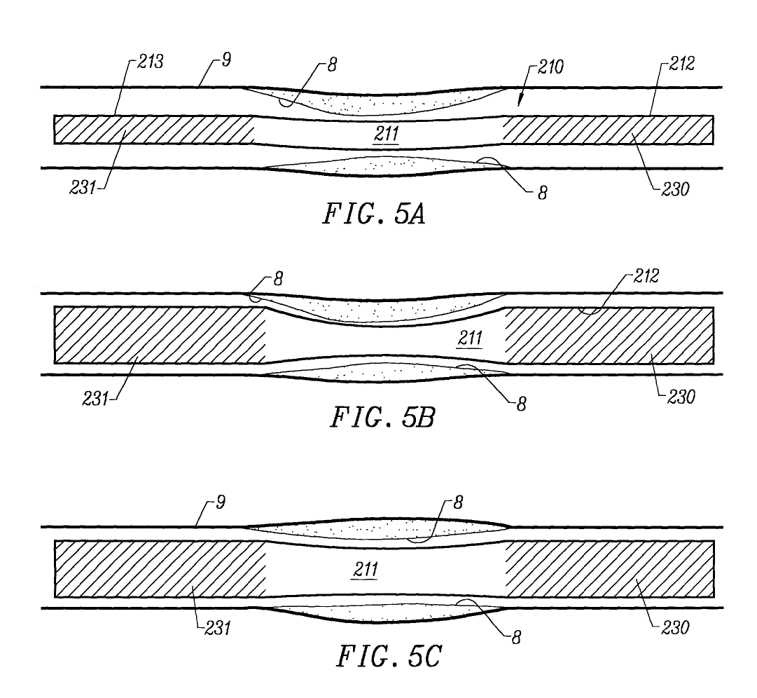

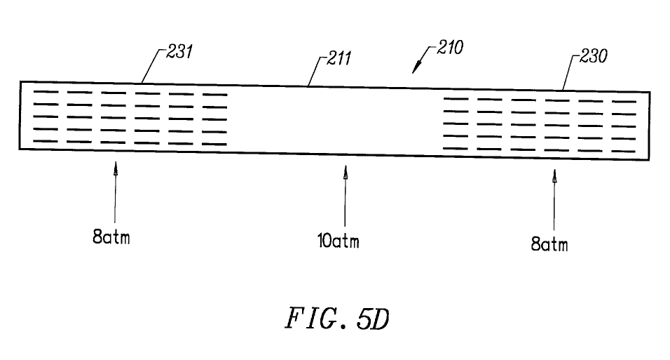

FIGS. 5A-5D illustrate how the present invention may be utilized to cause a stent to expand at its distal and proximal ends before it expands in its center region. A stent 210 is shown positioned in an artery 9 adjacent plaque deposit 8. The central region 211 of stent 210 is positioned adjacent the plaque deposit 8. The central region 211 of stent 210 has no relief cuts formed therein. The distal region 212 of stent 210 has a plurality of relief cuts shown generally as 230 formed therein. Similarly, the proximal end 213 of stent 210 has a plurality of relief cuts 231 formed therein. For the sake of illustration purposes, the relief cuts 230 and 231 in FIGS. 5A-5D are simply shown as cross hatching or dashed lines. It is to be understood that stent 210 may be any type of balloon expandable stent, including stent cell configurations illustrated in FIGS. 1-3 as well as a variety of other known stent configurations. For example, The Stenter's Notebook published by Physicians' Press in 1998 and written by Paul S. Phillips, M. D., Morton J. Kern, M. D. and Patrick W. Serruys, M. D. illustrates a variety of commercial, balloon expandable stents at pages 181-206. Those pages are herein incorporated by reference as though set forth in full herein. It is to be understood that the relief cuts according to the present invention may be utilized in any of the balloon expandable stent designs illustrated in The Stenter's Notebook. Some of those stent designs do not use a plurality of closed cells, but rather use single wire shapes formed in a variety of ways. Each of those designs has the common feature that a portion of the stent is designed to flex or bend to allow the stent to expand. It is within the scope of the present invention to apply the relief cuts of this invention to any of those prior art balloon expandable stent designs, preferably at the points of those stent designs where the maximum flexing and bending are designed to occur to allow the stent to expand. The invention may also be used in self-expanding stents, as discussed below.

The stent 210 illustrated generically in FIG. 5A is intended to include any of the stent configurations illustrated in The Stenter's Notebook, which are balloon expandable as well as those configurations illustrated in FIGS. 1-3 herein, and those shown and described in parent application Ser. No. 09/357,699, incorporated herein by reference as though set forth in full. With the relief cuts formed at the distal and proximal ends 230 and 231, respectively, as the balloon (not shown for clarity) expands, the distal and proximal ends 230 and 231 expand first as illustrated in FIG. 5B. This feature can be very important since, as illustrated in FIG. 5B, the distal portion 230 and proximal portion 231 of the stent will contact the vessel wall 9 before the central region of the stent 211 contacts the plaque deposit 8, thereby tending to “trap” the plaque deposit in its present position. As illustrated in FIG. 5C, the central region 211 of the stent which does not have any relief cuts requires somewhat additional pressure to expand and is shown in its expanded position where it contacts plaque deposit 8 and expands the restricted part of the artery. FIG. 5D illustrates typical balloon pressures; the distal and proximal ends 230,231 expand with 8 atm pressure and central section 211 expands at 10 atm pressure. Other expansion pressures can be used; FIG. 5D is presented only as an example.

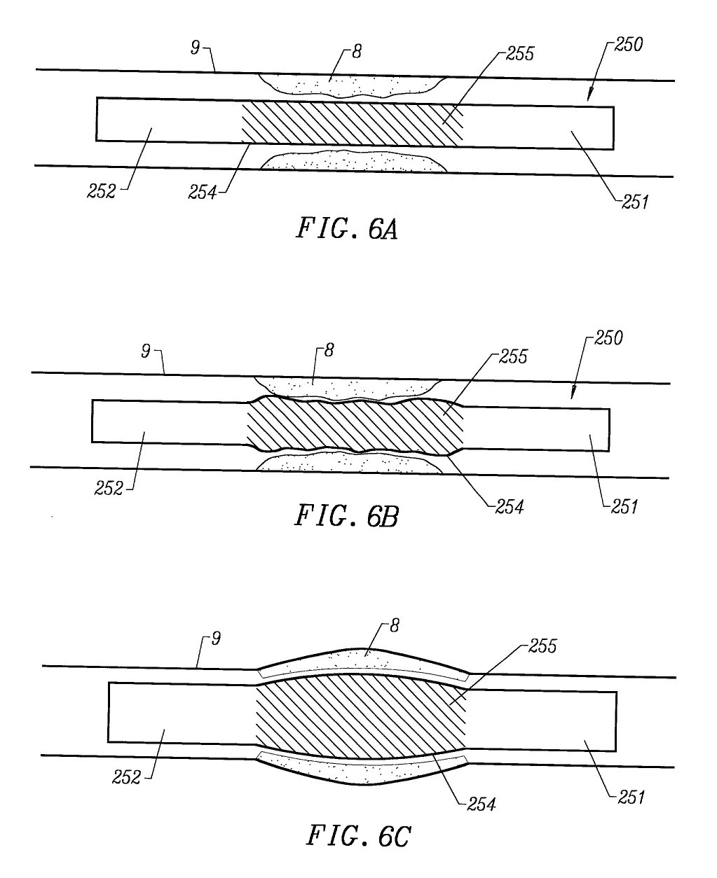

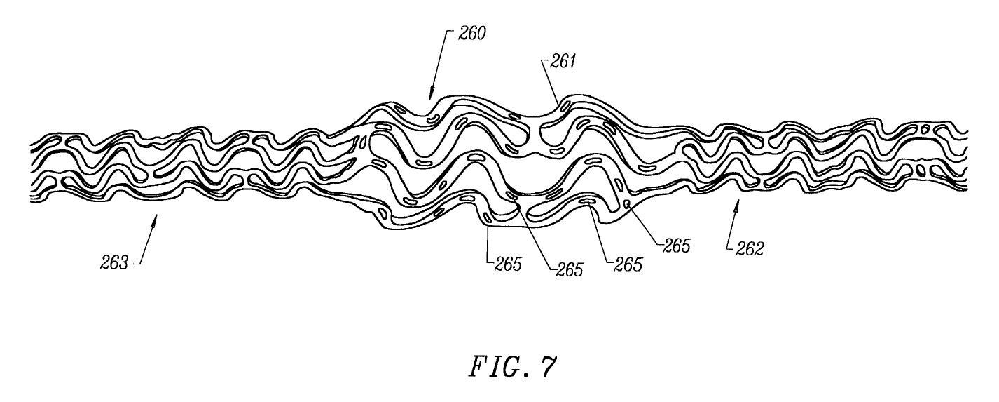

FIGS. 6A-6C illustrate a stent configuration 250 having no relief cuts at its distal end 251 or at its proximal end 252. Stent 250 does have a plurality of relief cuts 255 according to the present invention at its central region 254. As shown in FIG. 6B, the central region 254 having the relief cuts expands first. This feature can be advantageous in preventing longitudinal motion of the stent relative to the artery as it expands.

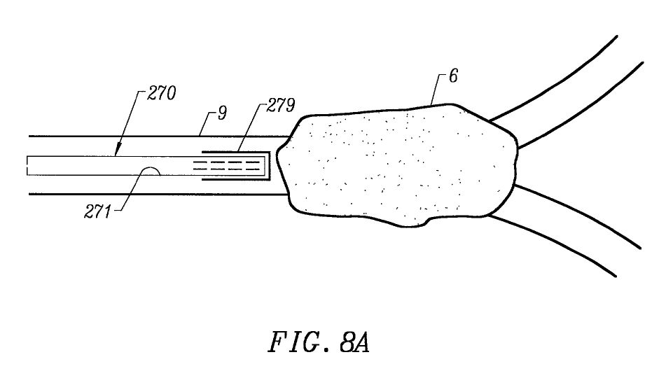

FIG. 7 illustrates yet another sinusoidal stent configuration 260 with relief cuts 265 formed only in its central region 261. That central region 261 will expand prior to the distal and proximal regions 262 and 263, respectively.

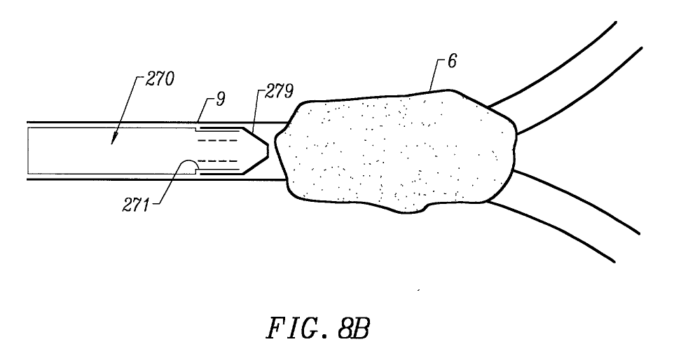

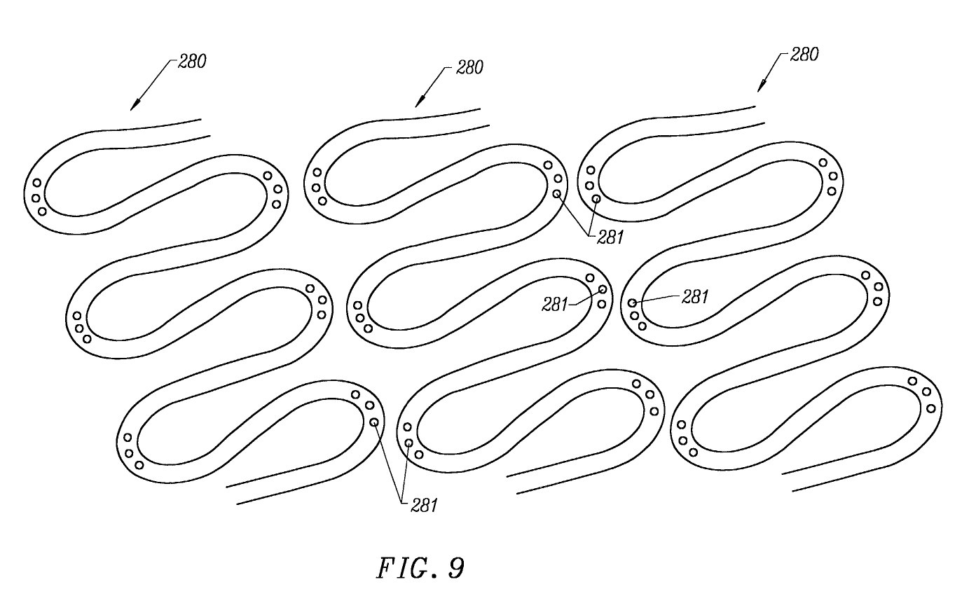

FIGS. 8A-8B illustrate how the present invention may be utilized to starve a cancerous tumor 6 fed by an artery 9. A stent 270 is placed in the artery close to the cancerous tumor 6. Stent 270 has relief cuts formed only in its distal end 271. Stent 270 carries an impermeable covering sheath 279 and, as its distal end 271 expands and contacts the walls of artery 9, blood flow through artery 9 to tumor 6 is interrupted, causing the tumor to die.

FIG. 9 illustrates how the present invention may be used with a continuous, single strand serpentine stent 280 known in the art. This type of stent is widely used in the art and is illustrated separately since it does not use a closed cell design but, nevertheless, may benefit significantly from the present invention. Relief cuts 281 are formed in serpentine stent 280 at the intended points of maximum flex or bending as the stent is expanded. Using the relief cuts as illustrated in FIG. 9 will allow the stent to expand in response to less balloon pressure. It is to be understood that the serpentine stent design could also be modified by increasing the width of the serpentine stent member and decreasing the thickness, in similar fashion as illustrated above in FIGS. 4D, 4E and 4G relative to the prior art closed cell diamond-shaped cell configuration.

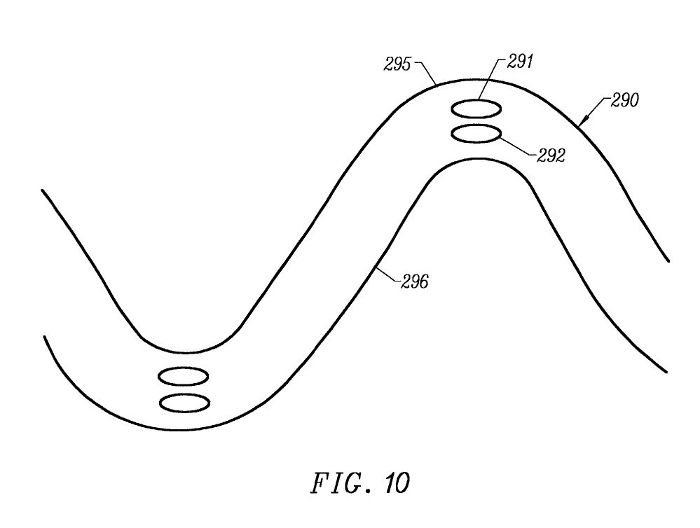

FIG. 10 illustrates another variation of the invention wherein stent 290 has relief cuts 291 and 292 formed near apex 295. In the embodiment illustrated in FIG. 10, relief cuts 291 and 292 are aligned transversely across the width of stent member 296. This embodiment of the invention is particularly useful for stents used in larger blood vessels and other body lumens wherein adequate width is available to position the relief cuts transversely.

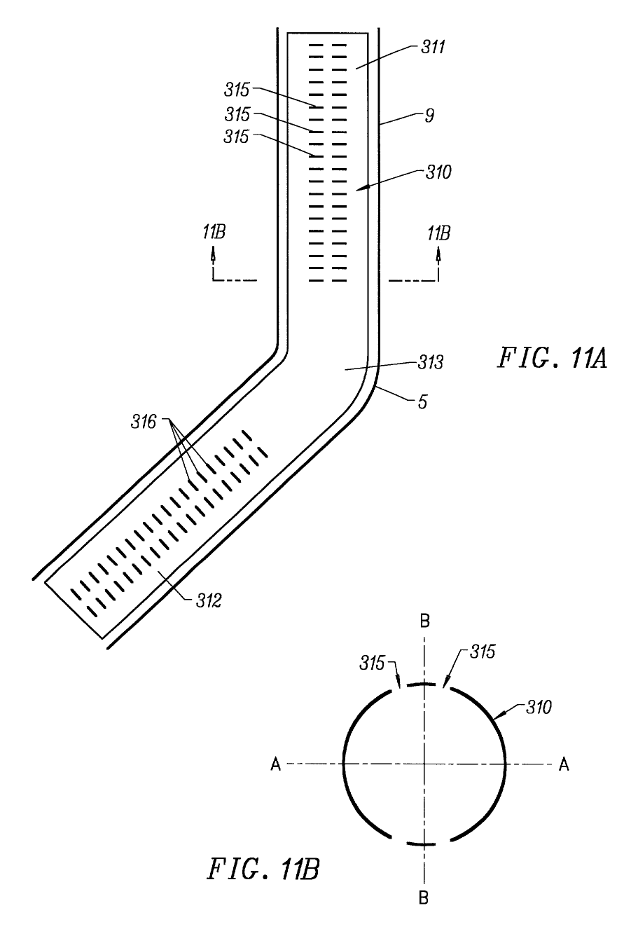

FIGS. 11-14 illustrate various techniques by which the relief cuts of the present invention may be used advantageously in curved or angulated arteries or other body lumens. For example, FIG. 11A illustrates an artery 9 having a curved region 5. Artery 9 is assumed to lie in a plane parallel with the drawing. Stent 310 is shown positioned in artery 9 in its expanded position. Stent 310 has a distal section 311, a proximal section 312 and a central section 313. The distant and proximal sections each carries a pattern of relief cuts illustrated by dashed lines 315 and 316, respectively. Central section 313 of stent 310 does not have relief cuts formed in it in the embodiment illustrated in FIG. 11A. The relief cuts 315 and 316 are not formed around the entire periphery of stent 310, as illustrated in sectional view 11B. As shown in sectional view 11B, relief cuts 315 are formed in the upper part and lowermost part of stent 310; however, no relief cuts are formed near the horizontal axis A illustrated in FIG. 11B. The effect of placing relief cuts as illustrated in FIG. 11B is to allow stent 310 to bend easily relative to vertical axis B-B in order to accommodate the curved section 5 of artery 9 to facilitate deployment of stent 310. The pattern of relief cuts as illustrated in FIGS. 11A and 11B tends to maximize the strength of stent 310 in its central region 313 to resist the crumpling of the curved region 5 of artery 9.

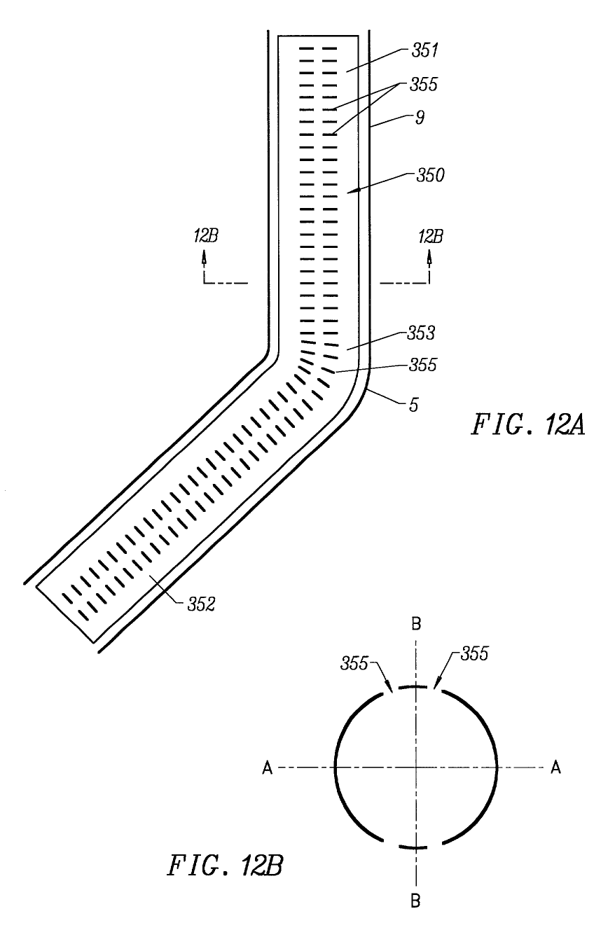

FIGS. 12A and 12B illustrate a variation to the relief cut pattern shown in FIG. 11. Stent 350 illustrated in FIG. 12 has relief cuts 355 formed along its entire length including central region 353 as well as distal and proximal ends 351 and 352. Again, the relief cuts 355 are only formed away from the horizontal axis A-A illustrated in FIG. 12B. Placement of the relief cuts in this fashion allows the stent 350 maximum flexibility to bend about vertical axis B-B illustrated in FIG. 12B to facilitate its placement and deployment in the curved artery 9.

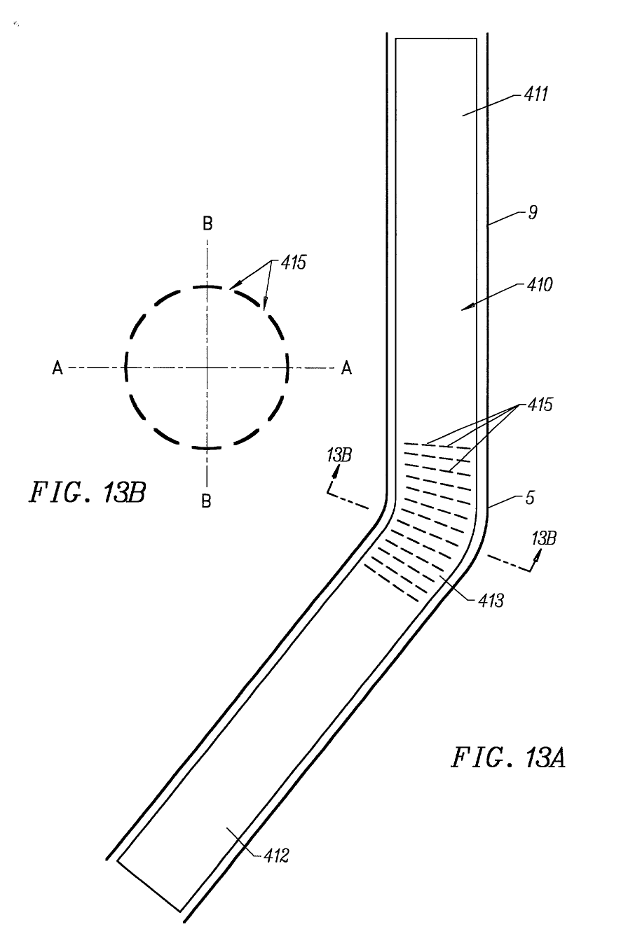

FIGS. 13A and 13B illustrate yet another manner in which relief cuts may be utilized to facilitate deployment of stent 410 in curved artery 9 having a curved section 5. In this embodiment, the pattern of relief cuts 415 is only formed in the central region 413 of stent 410. The proximal end and distal end 411,412 have no relief cuts formed therein. Central section 413 has relief cuts formed completely and uniformly around its periphery as illustrated in sectional view 13B. Placement of relief cuts around the entire periphery of the central region 413 allows maximum flexibility of stent 410 in the region where stent 410 must bend to conform to the curved region 5 of artery 9.

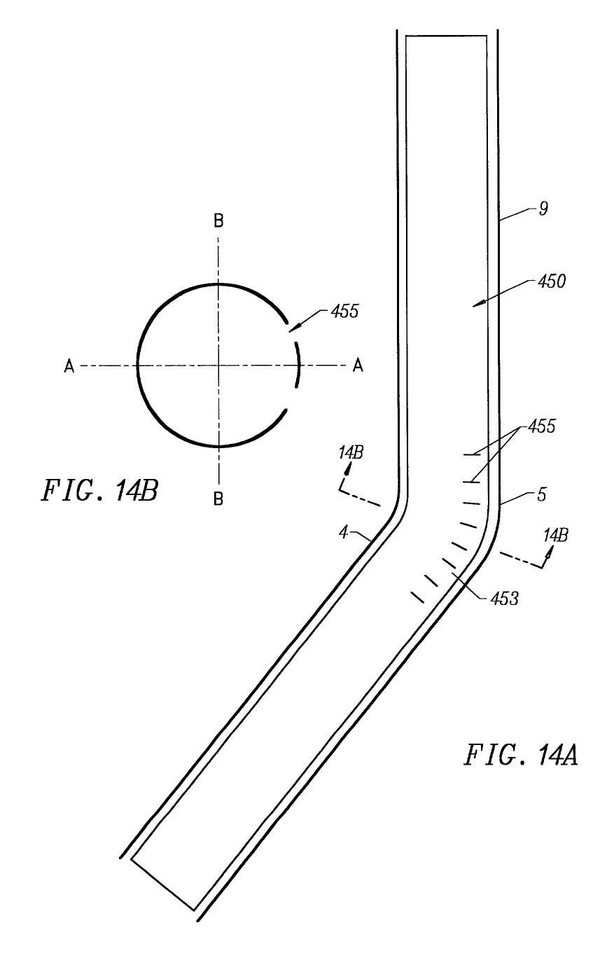

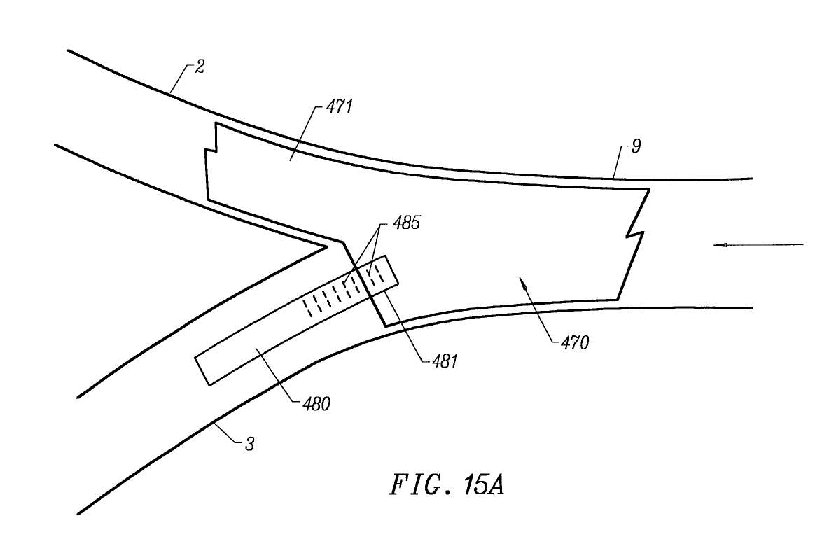

FIGS. 14A and 14B illustrate stent 450 having a pattern of relief cuts 455 placed only at the central region 453 of stent 450 and only in that portion of the stent periphery which contacts the outside radius of the arterial wall at the curved section 5. As illustrated in FIG. 14B, relief cuts 455 are placed radially outwardly of the vertical axis B-B of FIG. 14B and no relief cuts are formed on the inside radius, that is, radially inwardly of the central vertical axis B-B of stent 450. The purpose of placing relief cuts in this fashion is to allow the central portion 453 of stent 450 to flex to conform to the curved portion 5 of artery 9 while simultaneously allowing the stent to remain as rigid as possible adjacent the more sharply curved arterial wall region 4 which occurs at the radial inwardly side of the curved section of artery 9. The stent is therefore strongest along the inside radius 4 of curved artery 9, which is the part of the artery most likely to crimp.

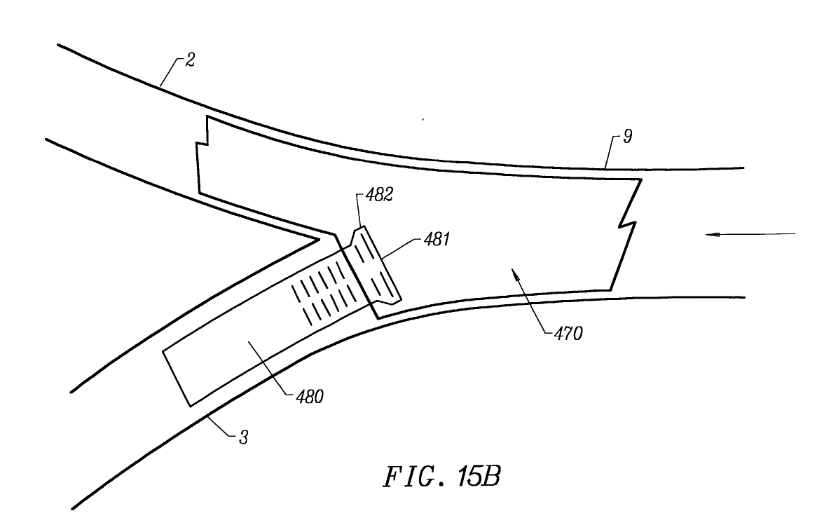

FIGS. 15A and 15B are schematic illustrations showing how the present invention can be used in conjunction with bifurcated stents. A bifurcated artery 9 splits into two branches 2 and 3. A first prior art stent 470 is placed in artery 9 and has an extension 471 that extends partially into branch 2. A second stent 480 is provided having a series of relief cuts 485 formed in its distal end 481. The distal end 481 of stent 480 is positioned inside stent 470 prior to being expanded. As shown in FIG. 15B, as stent 480 is expanded, its distal end 481 forms a “flare” 482 which effectively seats against stent 470 and which prevents stents 470 and 480 from separating after being deployed.

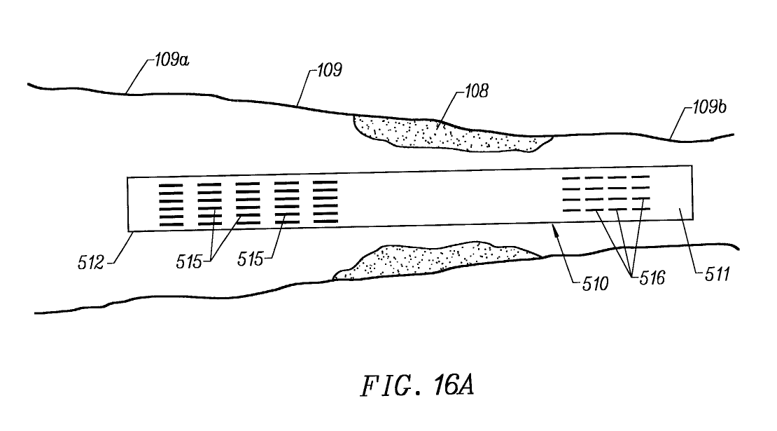

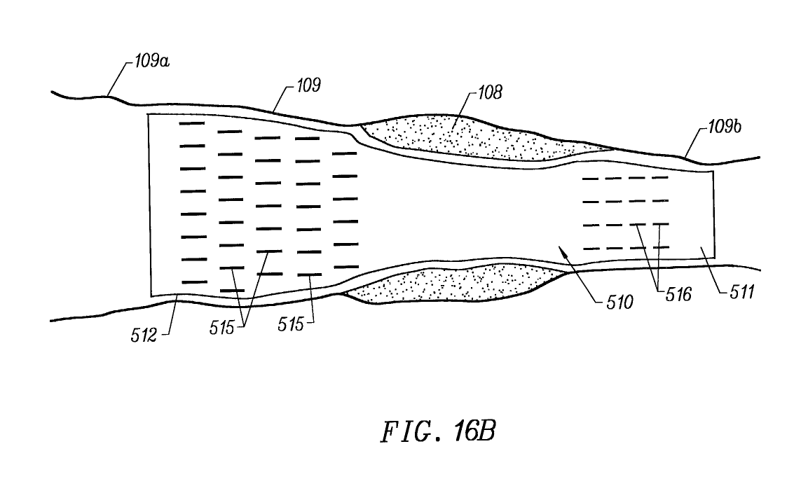

FIGS. 16A and 16B are schematic illustrations showing how the present invention can be utilized in arteries or other lumens having somewhat irregular shapes. Artery 109 is shown having a first section 109a of rather large diameter and a second section 109b having a somewhat reduced diameter. Plaque deposit 108 is illustrated in the generally tapered region of artery 109. A stent 510, which can be any prior art stent or any stent shown and described in the parent application referred to above. Stent 510 has a distal end 511 and a proximal end 512. A rather large number of relief cuts 515 are formed in proximal end 512 of stent 510. A somewhat smaller number of relief cuts is formed in the distal end 511 of the stent 510. The purpose of placing these patterns of relief cuts on stent 510 is to cause the stent 510 in its expanded position to conform as closely as possible to the walls of the artery 109 and the plaque deposit 108. As shown in FIG. 16B, the proximal end 512 expands further because of the presence of a greater number of relief cuts 515. The distal end 511 expands a somewhat reduced amount because of the absence of relief cuts. The small pattern of relief cuts 516 formed near the distal end 511 causes a somewhat greater expansion of stent 510 in that region to conform to the shape of the arterial wall and plaque deposit 108. FIGS. 16A and 16B illustrate how patterns of relief cuts can be utilized to make a stent expand in a controlled non-uniform fashion to conform to a somewhat irregularly shaped arterial wall. The stent in its expanded form effectively supports the irregularly shaped vessel wall and plaque deposits and simultaneously seals off any leaks that would otherwise occur at the proximal and distal ends of the stent.

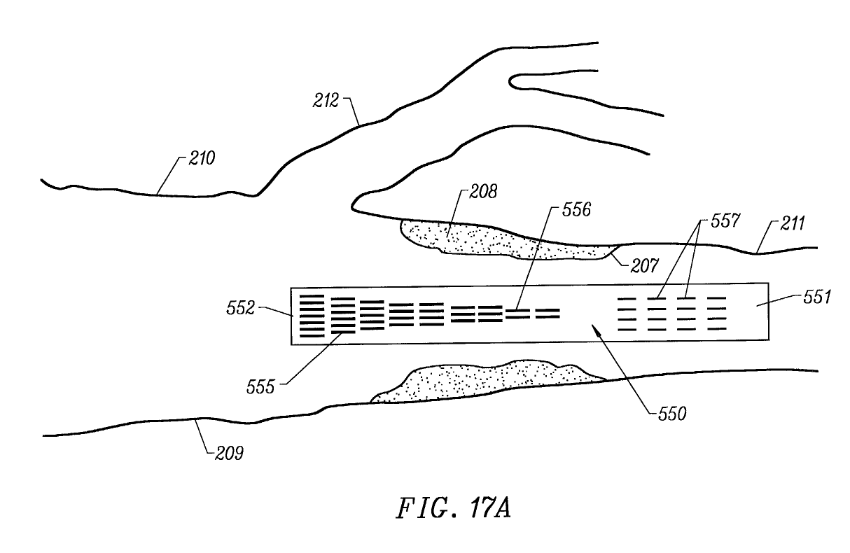

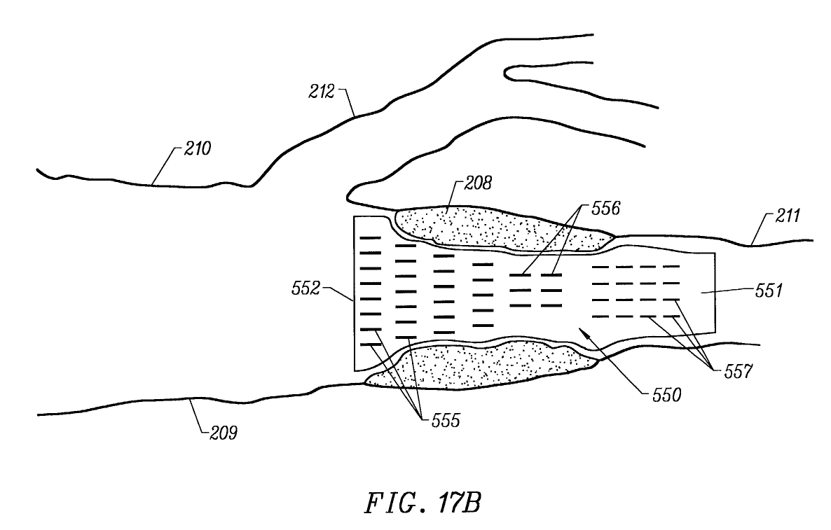

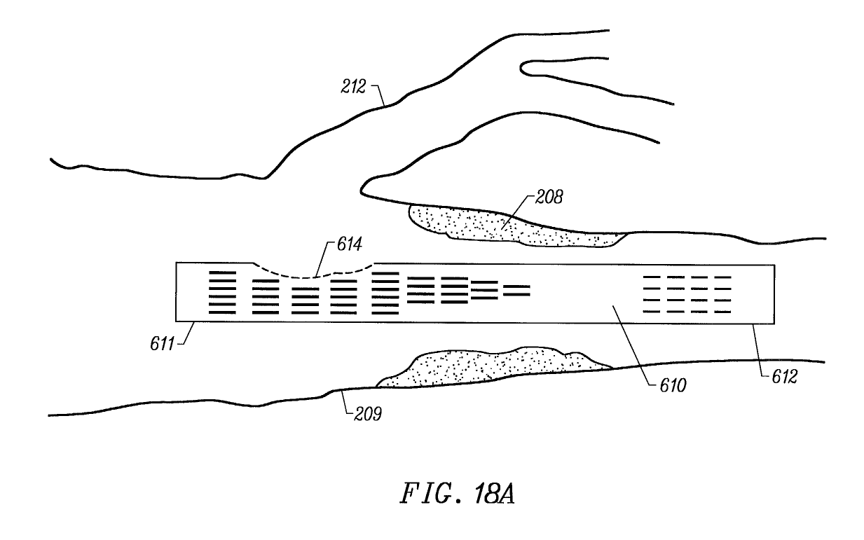

FIGS. 17A and 17B are schematic illustrations showing how the present invention may be utilized in an artery or other lumen having a branch artery at or near the location where the stent is to be deployed. Artery 209 has a first region 210 of relatively large diameter and a second downstream region 211 having a considerably reduced diameter. A branch artery 212 connects to artery 209 near a plaque deposit 208. Stent 550 is provided having a distal end 551 and a proximal end 552. Since the proximal end 552 must expand a greater distance than the distal end 551, a relatively large number of relief cuts 555 is placed near proximal end 552. The number of relief cuts is gradually reduced and, at the center of stent 550, a relatively sparse pattern 556 of relief cuts is applied where the stent should be expanded the least. Towards the distal end of stent 551 a secondary pattern 557 of relief cuts is applied so that the stent may expand to a somewhat greater degree adjacent the distal end 207 of plaque deposit 208. Stent 550 is shown in its expanded form in FIG. 17B and it can be seen that the patterning of relief cuts allows the stent to expand in a controlled non-uniform fashion to conform to the walls of the artery 209 and to achieve the desired blood flow through artery 209.

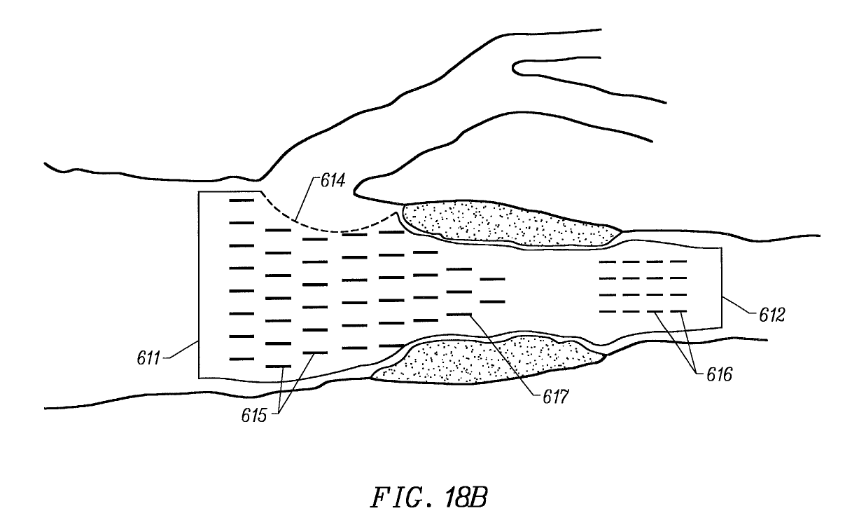

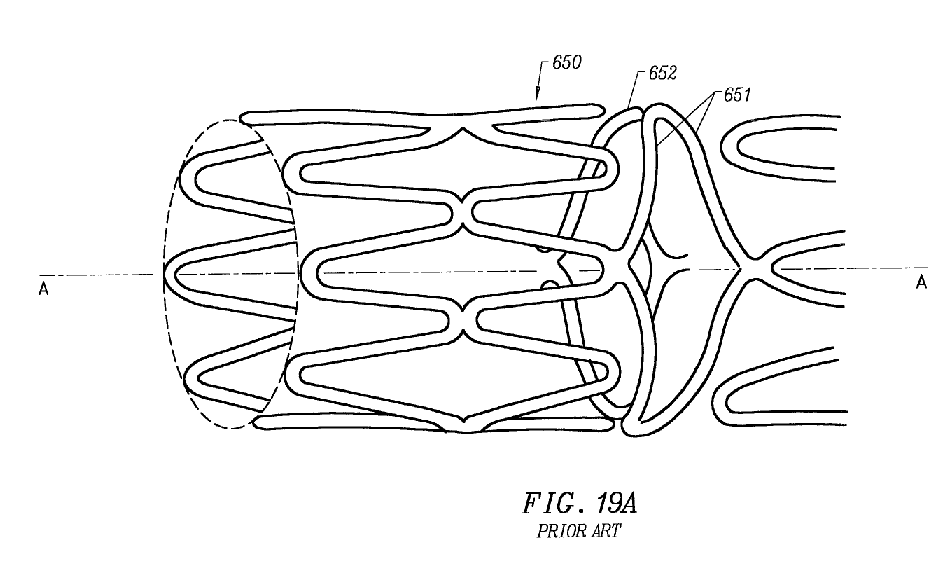

FIGS. 18A and 18B show an alternate stent design 610 which may be utilized in the irregular shaped artery 209 illustrated in FIGS. 17A and 17B with plaque deposit 208 and branch artery 212. Stent 610 has a proximal end 611 and a distal end 612. The proximal end of stent 610 extends beyond the location where branch artery 212 connects with artery 209. Stent 610 has an opening 614 formed in its surface adjacent where stent 610 will expand against the base of branch artery 212. The opening 614 in stent 610 allows blood to flow freely from artery 209 into branch artery 212. Stent 610 has a greater number of relief cuts 615 formed at its proximal end 611 as compared to the relief cuts 616 formed at its distal end. A tapering pattern of relief cuts 617 is formed in the center of stent 610 to allow the stent to conform to the required taper. As shown in FIG. 18B, the relief cut patterns are designed to allow the stent to expand in a controlled, non-uniform manner to conform to the wall of the artery (or other lumen) and to prevent end leaks.

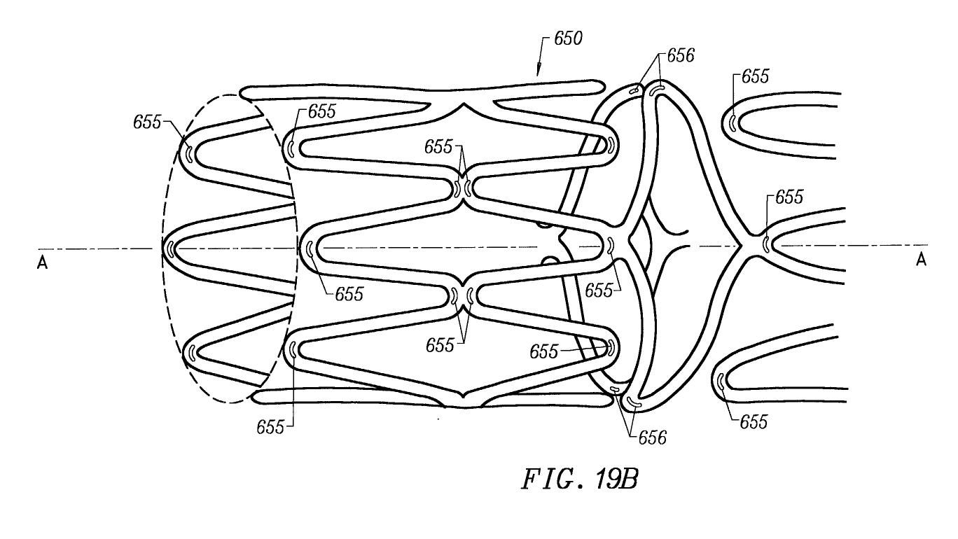

FIG. 19A illustrates a prior art stent design 650 shown in U.S. Pat. No. 6,159,237, wherein the stent includes transverse cells 651 and 652. As stent 650 expands, cells 651 and 652 expand along the longitudinal axis A-A of the stent 650.

FIG. 19B shows the relief cuts of the present invention as applied to the stent design 650 of FIG. 19A. Relief cuts 655 allow stent 650 to expand radially in a first direction to increase its circumference. Relief cuts 656 in transverse cells 651 and 652 allow cells 651 and 652 to expand more easily in a second direction parallel to longitudinal axis A-A. FIG. 19B illustrates how the present invention can be used to facilitate stent cell expansion in two directions (longitudinally and circumferentially) simultaneously. Alternately, relief cuts 656 in the transverse cells may be utilized without using relief cuts 655; this variation would allow the transverse cells to expand more easily than the circumferentially expanding cells. Also, relief cuts 655 may be used without using relief cuts 656.

Although the above description of the invention has concentrated on balloon expandable stents, the invention is also useful with self-expanding stents. In the case of self-expanding stents, the use of relief cuts allows the use of flatter and thinner walled stents, increasing the radio-opacity and vessel wall coverage of the stent. Furthermore, using patterns of relief cuts in self-expanding stents, those stents may be caused to expand in a controlled, non-uniform fashion which can be advantageous in many situations. Each of the figures illustrated herein, including the various stent cell configurations and the various patterns of relief cuts illustrated in the drawings, may all be applied to self-expanding stents.

The present invention is usable with stents made of any material such as nitinol, stainless steel, plastic and composite materials.

The foregoing description of the invention has been presented for purposes of illustration and description and is not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is defined by the following claims.

What is claimed is:

1. In an expandable stent, wherein said stent has a plurality of interconnected members, and wherein said interconnected members flex relative to each other as said stent expands, the improvement comprising:
an array of relief cut means formed in some of said interconnected members whereby said members flex more easily than without said relief cut means, and wherein each of said relief cut means is sufficiently small that the columnar compressive strength of said interconnected members is not significantly reduced by the presence of said relief cut means formed therein.

2. The apparatus of claim 1 wherein said stent is a balloon expandable stent.

3. The apparatus of claim 2 wherein said stent has distal and proximal ends and a central section, and wherein said relief cut means are formed only in said distal and proximal ends.

4. The apparatus of claim 2 wherein said stent has distal and proximal ends and a central section, and wherein said relief cut means are formed only in said central section.

5. The apparatus of claim 2 wherein said interconnected members of said stent have cross sections wherein the width is greater than the thickness.

6. The apparatus of claim 5 wherein said width is between 1.5 and 5 times as great as said thickness.

7. The apparatus of claim 1 wherein said relief cut means comprises one or more holes formed in and extending through one or more of said interconnecting members.

8. The apparatus of claim 7 wherein said holes are circular.

9. The apparatus of claim 7 wherein said holes are elliptical.

10. The apparatus of claim 7 wherein said holes are slots.

11. The apparatus of claim 1 wherein said stent is a self-expandable stent.

12. The apparatus of claim 1 wherein said relief cuts are applied to said stent in patterns to allow controlled, non-uniform expansion of said stent.

13. In an expandable stent having a plurality of cells and said stent is movable between a retracted and an expanded position, wherein said cells are formed by a plurality of flexible, interconnected members, said members flexing relative to each other as said tent expands, the improvement comprising:
an array of relief cut means formed in some of said members to cause said members to flex more easily than without said relief cut means being formed therein.

14. The apparatus of claim 13 wherein said stent is a balloon-expandable stent.

15. The apparatus of claim 13 wherein said interconnected members have cross sections wherein the width is between 1.5 and 5 times as great as the thickness.
